(19) United States
(12) Patent Application Publication
(10) Pub. No.: US 2026/0176610 A1
(43) Pub. Date: Jun. 25, 2026

(54) COMPOSITE BIOCHAR IMMOBILIZED MICROBIAL FILLER AND ITS APPLICATION IN THE DEGRADATION OF VOLATILE ORGANIC POLLUTANTS

(71) Applicant: ZHEJIANG UNIVERSITY OF TECHNOLOGY, Hangzhou (CN)

(72) Inventors: Zhuowei CHENG, Hangzhou (CN); Zhirong SUN, Hangzhou (CN); Jianming YU, Hangzhou (CN); Weiyue ZHANG, Hangzhou (CN); Jiade WANG, Hangzhou (CN); Dongzhi CHEN, Hangzhou (CN); Jianmeng CHEN, Hangzhou (CN)

(21) Appl. No.: 18/999,491

(22) Filed: Dec. 23, 2024

(30) Foreign Application Priority Data
Dec. 25, 2023 (CN) 202311789140.7

(51) Int. Cl. C12N 11/093; B01D 53/72; B01D 53/85; C12N 1/20; C12N 11/14; C12R 1/00

(52) U.S. Cl. CPC C12N 11/093; B01D 53/72; B01D 53/85; C12N 1/205; C12N 11/14; C12R 2001/00

(57) ABSTRACT

The present invention discloses a composite biochar immobilized microbial filler and its application in the degradation of volatile organic pollutants. The composite biochar immobilized microbial filler utilizes bamboo fiber, thermoplastic polyurethane, and bamboo biochar as the carrier, and is made with efficient degradation bacterial suspension for volatile organic pollutants as the active microorganism. The composite biochar immobilized microbial filler prepared by the present invention has advantages such as high surface activity, high mechanical strength, high degradation efficiency, and high microbial immobilization capacity. It enhances the adsorption and degradation ability of microorganisms towards VOCs while simultaneously improving the mechanical strength of the immobilized microbial filler, thereby addressing challenges in biological purification technology. The biochar immobilized microbial filler of this invention is used for the degradation of volatile organic pollutants, wherein the polyurethane and bamboo material are inexpensive and easily obtainable, and the preparation process is simple and efficient, offering significant economic cost advantages.

### TECHNICAL FIELD

The present invention relates to a composite biochar immobilized microorganism and its application in the degradation of volatile organic pollutants.

### BACKGROUND OF THE INVENTION

Currently, the pollution caused by volatile organic compounds (VOCs) emitted from industrial emissions and the treatment of “three wastes” is severe. Human-induced sources, such as manufacturing, petrochemical, and pharmaceutical industries, emit a large amount of VOCs, posing serious threats to the environment and human health. The treatment of VOCs requires the rational utilization of resources, cost reduction, and improvement of the efficiency of the entire treatment process.

Biodegradation is a green process that utilizes microbial adsorption metabolism and enzyme catalysis to remove pollutants. Biodegradation-based treatment technologies are generally more effective than other VOCs control technologies. With proper process design and operational procedures, biodegradation technologies exhibit excellent treatment performance, low treatment costs, and friendly ecological benefits. However, biodegradation is hindered by many factors, including the growth and separation of microbial cells, mass transfer of pollutants, toxicity, and adverse environmental conditions, which lead to reduced activity and limit its application in production and promotion. Cell immobilization technology can effectively address the aforementioned issues. However, traditional microbial immobilization carriers, such as organic carriers like polyurethane, polyvinyl alcohol, alginate, and inorganic carriers like SiO2, ceramics, and carbon materials, face challenges in terms of mechanical properties, mass transfer efficiency, and cost.

Therefore, it is generally believed that finding a carrier that possesses adsorption, degradation, and mechanical strength during the immobilization process is crucial.

### BRIEF SUMMARY OF THE INVENTION

The purpose of this invention is to provide a composite biochar immobilized microbial filler and its application in the degradation of volatile organic pollutants. This invention utilizes bamboo fiber, thermoplastic polyurethane, and biochar as a carrier to immobilize microorganisms, and the resulting composite biochar immobilized microbial filler is not only suitable for microbial growth but also possesses good mechanical strength, exhibits both adsorption and degradation capabilities for volatile organic pollutants, and is particularly suitable for the aerobic degradation of refractory VOCs, enhancing degradation efficiency and reducing costs.

The technical solutions adopted in this invention are as follows:

The present invention provides a composite biochar immobilized microbial filler enhanced by bamboo fiber and thermoplastic polyurethane, which is made with bamboo fiber, thermoplastic polyurethane, and bamboo biochar as the carrier, and an efficient degrading bacterial suspension for volatile organic pollutants as the active microorganism; wherein the mass ratio of bamboo biochar to bamboo fiber and thermoplastic polyurethane is 5:1-4:1-4; the OD₆₀₀ of the efficient degrading bacterial suspension is 0.1-1.0, and the volume usage is 100-250 mL/g based on mass of the carrier.

Furthermore, the mass ratio of bamboo biochar to bamboo fiber and thermoplastic polyurethane is 5:2:3, the OD₆₀₀ of the efficient degrading bacterial suspension is 0.5, and the volume usage is 125 mL/g based on the mass of the carrier.

Furthermore, the efficient degrading bacterium is Acinetobacter venetianus PFZR-1, which degrades cyclohexane; it has a white, aerobic, smooth, and moist colony with an ellipsoidal shape; it is deposited at the China Center for Type Culture Collection (CCTCC) with the deposit number CCTCC NO: M 2022719 and the deposit date of May 24, 2022, the address is Wuhan University, Wuhan, China, 430072; and it has been disclosed in patent application CN115786183A.

Furthermore, the efficient degrading bacterial suspension is obtained by washing the wet bacterial cells obtained from fermentation culture with a buffer solution and resuspending them with sterile water, the OD₆₀₀ of the efficient degrading bacterial suspension ranges from 0.1 to 1.0, with a more preferred OD₆₀₀ of 0.5. The preparation of the bacterial suspension follows the following steps: inoculating the efficient degrading bacteria into LB solid medium, culturing them at 30° C. for 1 day, then inoculating them into a fermentation medium, incubating them at 30° C. and 160 rpm for 48 hours, after centrifugation, collecting the wet bacterial cells, washing them with a buffer solution (preferably washing for three times in pH 7, 0.05 M phosphate buffer solution (PBS)), and then resuspending them with sterile water to obtain the bacterial suspension. The LB solid medium is composed of: 5 g/L yeast extract, 10 g/L NaCl, 10 g/L peptone, 15-20 g/L agar, pH natural, and deionized water as the solvent. The fermentation medium is composed of: 5 g/L yeast extract, 10 g/L NaCl, 10 g/L peptone, pH natural, and deionized water as the solvent.

Furthermore, the bamboo fiber is prepared using the following method: soaking the bamboo fiber feedstock in distilled water at room temperature for 6-12 hours, washing it with distilled water (preferably for three times), then adding distilled water to the soaked material and pouring the mixture into a beater for beating, taking the slurry with a beating degree of 30-70° SR and drying it to obtain bamboo fiber; wherein the volume of distilled water used for soaking is 5-15 mL/g based on the mass of the bamboo fiber-like raw material, preferably 10 mL/g; the volume of distilled water used for beating is 1-10 mL/g based on the mass of the bamboo fiber-like raw material, preferably 5 mL/g.

Furthermore, the bamboo biochar is prepared using the following steps: placing the absolutely dry bamboo material (preferably bamboo fiber prepared by the aforementioned method) in a porcelain boat in a tube furnace, under a nitrogen atmosphere, increasing the temperature to 500-700° C. at a rate of 2-6° C./min and maintaining it for 1-3 hours (preferably with a nitrogen flow rate of 30 L/min, increasing the temperature to 600° C. at 5° C./min and maintaining it for 2 hours), stopping nitrogen supply, then using water vapor to increase the temperature to 600-1000° C. at a rate of 1-4° C./min and maintaining it for 1-3 hours (preferably increase the temperature to 800° C. at 2.5° C./min and maintain it for 2 hours), cooling the material to room temperature, and sieving it through an 80-100 mesh screen to obtain bamboo biochar.

Furthermore, the selected thermoplastic polyurethane is in the form of transparent particles, with a molecular weight of 90K, a softening temperature of 100° C., and a melting point of 180° C.

The present invention further provides a method for preparing the composite biochar immobilized microbial filler, which comprises the following steps:
(1) dissolving the thermoplastic polyurethane in N,N-dimethylformamide with stirring at room temperature (preferably at a stirring speed of 500 r/min for 2 hours to ensure complete dissolution) to yield a polyurethane solution; dispersing bamboo biochar and bamboo fiber into the polyurethane solution, stirring at room temperature for 1-3 hours (preferably 2 hours), then adding deionized water, allowing the mixture to stand for precipitation at room temperature (preferably for 2 hours), then taking the precipitate, pressing it into shape (preferably into a paper-like form), and drying it to obtain a carrier;
(2) immersing the carrier from step (1) in the efficient degrading bacterial suspension, and subjecting it to immobilization on a shaker at 25-35° C. and 100-200 r/min for 20-30 hours; after removal, drying it at room temperature to obtain the composite biochar immobilized microorganism.

Furthermore, in step (1), the volume of N,N-dimethylformamide used is 10-15 mL/g, preferably 12.5 mL/g, based on the mass of polyurethane; the volume of deionized water used is 50-275 mL/g, preferably 240 mL/g, based on the mass of polyurethane.

Furthermore, in step (1), pressing into shape and drying are carried out as follows: the precipitate is manufactured on the sheet former at 100° C. for 10 minutes, and then placed in an oven and dried at 80° C. for 12 hours to obtain a paper sheet.

Furthermore, step (2) involves immobilization for 24 hours at 30° C. and 160 r/min on a shaker.

The present invention further provides an application of the composite biochar immobilized microbial filler in the degradation of one or more volatile organic pollutants, and the application method is as follows: using the composite biochar immobilized microbial filler as the catalyst, one or more volatile organic pollutants as the substrate, and an inorganic salt culture medium as the reaction medium, carry out the reaction under conditions of a pH range of 5-8 and a temperature range of 25-35° C., to achieve the degradation of volatile organic pollutants.

Preferably, the reaction is carried out at pH 7 and 30° C.

Preferably, the amount of the catalyst used is 2-6 g/L, preferably 4 g/L, based on the volume of the inorganic salt culture medium; the concentration of the volatile organic pollutant is 150-300 mg/L, preferably 189.84 mg/L, based on the volume of the inorganic salt culture medium; and the volatile organic pollutant is cyclohexane.

Preferably, the inorganic salt medium is composed of: K₂HPO₄ 0.942 g/L, KH₂PO₄ 0.234 g/L, NaNO₃ 1.7 g/L, NH₄Cl 0.98 g/L, MgCl₂·6H₂O 0.2033 g/L, CaCl₂ 2H₂O 0.0111 g/L, FeCl₃ 0.0162 g/L, a trace element solution 5 mL/L, and deionized water as the solvent, with a pH of 7; wherein the trace element solution is composed of: ZnCl₂ 0.088 g/L, MnCl₂·4H₂O 0.060 g/L, KI 0.01 g/L, Na₂MoO₄·2H₂O 0.1 g/L, H₃BO₃ 0.05 g/L, and deionized water as the solvent.

The bamboo fiber (BF) of this invention possesses excellent characteristics such as renewability, biodegradability, biocompatibility, strong hydrophilicity, mechanical properties, and heat resistance. Bamboo biochar exhibits great potential for environmental remediation applications due to its high specific surface area, multi-level pore structure, and abundant and variable surface functional groups. Thermoplastic polyurethane (PU) has excellent mechanical properties and is a mature eco-friendly material. Therefore, synthesizing a new type of immobilized microbial filler by combining the advantages of bamboo materials and thermoplastic polyurethane can effectively solve the technical challenges of traditional biological purification.

The beneficial effects of the present invention are as follows:
1. This invention utilizes highly active bamboo biochar, microbe-friendly bamboo fiber, and thermoplastic polyurethane as carriers. It enhances the adsorption and degradation capabilities of microorganisms towards VOCs, while simultaneously improving the mechanical strength of the immobilized microbial filler, thus addressing the challenges in biological purification technology.
2. The composite biochar immobilized microbial filler prepared by this invention possesses several advantages, including high surface activity (adsorption capacity of 46.18 mg/g), high mechanical strength (tensile strength exceeding 7 Nm/g and burst strength exceeding 0.1 Kpa m2/g), high degradation efficiency (100% degradation efficiency at a concentration of 189.84 mg/L), and high microbial immobilization capacity (immobilization efficiency of 75.8%).
3. The biochar-immobilized microbial filler of the present invention is used for the degradation of volatile organic pollutants. The polyurethane and bamboo material are cheap and easily available, and the preparation process is simple and efficient, offering significant economic cost advantages.

### BRIEF DESCRIPTION OF THE DRAWINGS

FIG. 1 is a photo of the biochar-immobilized microbial filler prepared in Example 1, where A represents filler A; B represents filler B; C represents filler C; and D represents filler D.

FIG. 2 shows the strength test results of fillers with different raw material ratios; A represents a mass ratio of BF to PU of 4:1, B represents a mass ratio of BF to PU of 3:2, C represents a mass ratio of BF to PU of 2:3, and D represents a mass ratio of BF to PU of 1:4.

FIG. 3 presents a bar chart illustrating the microbial immobilization efficiency of fillers with varying raw material ratios; A represents a mass ratio of BF to PU of 4:1, B represents a mass ratio of BF to PU of 3:2, C represents a mass ratio of BF to PU of 2:3, and D represents a mass ratio of BF to PU of 1:4.

FIG. 4 presents scanning electron microscope images of the immobilized microbial fillers; A represents a mass ratio of BF to PU of 4:1, B represents a mass ratio of BF to PU of 3:2, C represents a mass ratio of BF to PU of 2:3, and D represents a mass ratio of BF to PU of 1:4.

FIG. 5 compares the degradation effects of immobilized fillers and free bacteria on cyclohexane.

### DETAILED DESCRIPTION OF THE INVENTION

The present invention will be further described below in conjunction with specific examples, but the scope of protection of the present invention is not limited thereto:

The raw materials used in the following examples are all commercially available products. Bamboo material was purchased from Zhejiang Jibamboo Biotechnology Co., Ltd. Thermoplastic polyurethane was purchased from Taiwan Risheng Co., Ltd., with a molecular weight of 90K, a softening temperature of 100° C., and a melting point of 180° C. Anhydrous ethanol was purchased from China National Pharmaceutical Group Co., Ltd. N,N-Dimethylformamide (DMF) was also purchased from China National Pharmaceutical Group Co., Ltd. The sheet former was purchased from Xianyang Tongda Light Industry Equipment Co., Ltd., model TD10-200.

The Acinetobacter venetianus PFZR-1, deposited at the China Center for Type Culture Collection, with the deposit number CCTCC NO: M 2022719, and the deposit date of May 24, 2022, located at Wuhan University, Wuhan, China, 430072, has been disclosed in patent application CN115786183A.

LB solid medium is composed of: 5 g/L yeast extract, 10 g/L NaCl, 10 g/L peptone, 15-20 g/L agar, pH natural, and deionized water as the solvent.

Fermentation medium is composed of: 5 g/L yeast extract, 10 g/L NaCl, 10 g/L peptone, pH natural, and deionized water as the solvent.

Inorganic salt medium is composed of: K₂HPO₄ 0.942 g/L, KH₂PO₄ 0.234 g/L, NaNO₃ 1.7 g/L, NH₄Cl 0.98 g/L, MgCl₂·6H₂O 0.2033 g/L, CaCl₂) 2H₂O 0.0111 g/L, FeCl₃ 0.0162 g/L, trace element solution 5 mL/L, and deionized water as the solvent, with pH 7; wherein the trace element solution is composed of: ZnCl₂ 0.088 g/L, MnCl₂ 4H₂O 0.060 g/L, KI 0.01 g/L, Na₂MoO₄·2H₂O 0.1 g/L, H₃BO₃ 0.05 g/L, and deionized water as the solvent.

The room temperature is 25-30° C.

### Example 1: Preparation of Composite Biochar-immobilized Microbial Filler

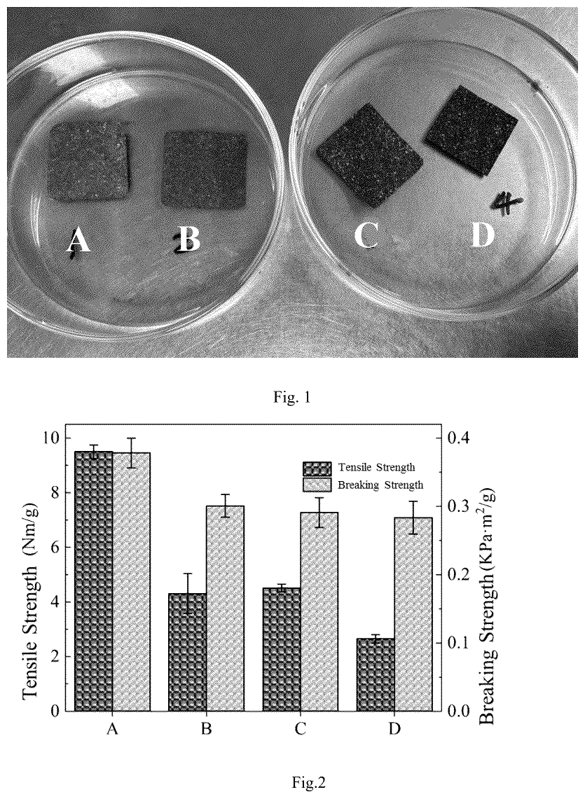

(1) Bamboo Fiber (BF): 460 g of bamboo material was soaked in 5 L of distilled water at room temperature for 12 hours. After being washed three times with distilled water, the soaked bamboo was added to 23 L of distilled water and then poured into a beater for beating. The slurry with a beating degree of 60° SR was obtained and dried at 80° C. for 12 hours to yield bamboo fiber. The beating degree was determined by taking a portion of the slurry, drying it to constant weight, dissolving 2 g of the absolutely dry slurry in 1000 mL of water, and measuring the beating degree in a Beating Degree Tester according to GB/T 3332-2004.
(2) Bamboo Biochar: 20 g of bamboo fiber prepared in step (1) was placed in a porcelain boat in a tube furnace. Nitrogen gas was introduced at a rate of 30 L/min. Under a nitrogen atmosphere, the temperature was increased to 600° C. at a rate of 5° C./min and held for 2 hours. Nitrogen gas was then stopped, and water vapor was used to increase the temperature to 800° C. at a rate of 2.5° C./min and held for another 2 hours. After cooling to room temperature, the material was sieved through an 80-100 mesh sieve to obtain 12 g of bamboo biochar.
(3) Efficient Degrading Bacterial Suspension: The Acinetobacter venetianus PFZR-1 was inoculated onto LB solid medium and cultured at 30° C. for 1 day. It was then inoculated into fermentation medium and cultured at 30° C. and 160 rpm for 48 hours. The culture was centrifuged to collect wet bacterial cells, which were washed three times with a pH 7, 0.05M phosphate-buffered saline (PBS) solution and then resuspended in sterile water to obtain a bacterial suspension with an OD600 of 0.5.
(4) 1 g of thermoplastic polyurethane (PU) was dissolved in 12.5 mL of N,N-dimethylformamide (DMF) (DMF/PU=12.5 mL/g) and stirred at room temperature and 500 r/min for 2 hours to obtain a polyurethane solution. 5 g of bamboo biochar prepared in step (2) and 4 g of bamboo fiber prepared in step (1) were dispersed into the polyurethane solution and stirred at room temperature for 2 hours. Then, 240 mL of deionized water was added, and the mixture was allowed to stand and precipitate at room temperature for 2 hours. The precipitated material was sheeted on a sheet former at 100° C. for 10 minutes to form a paper sheet with a diameter of 20 cm. The sheet was washed with clean water, placed in an oven, and dried at 80° C. for 12 hours. After equilibrium weighting, the carrier was obtained and labeled as Carrier A. Under the same conditions, Carriers B, C, and D were obtained by changing the amount of thermoplastic polyurethane to 2 g, 3 g, and 4 g, respectively, and adjusting the amount of bamboo fiber to 3 g, 2 g, and 1 g, respectively, with the other operations remaining the same.
(5) Preparation of Composite Biochar-immobilized Microbial Fillers: 0.2 g of Carrier A obtained in step (4) was immersed in 50 mL of the bacterial suspension obtained in step (3) and subjected to immobilization on a shaker at 30° C. and 160 r/min for 24 hours. After removal, it was dried at room temperature to obtain the composite biochar-immobilized microbial filler, labeled as Filler A (mass ratio of bamboo fiber to polyurethane was 4:1). See FIG. 1 for photo.

Under the same conditions, Carriers B, C, and D were substituted for Carrier A in step (5), and the other operations remained the same to obtain Fillers B (mass ratio of bamboo fiber to polyurethane was 3:2), C (mass ratio of bamboo fiber to polyurethane was 2:3), and D (mass ratio of bamboo fiber to polyurethane was 1:4), respectively. See FIG. 1 for photos.

### Example 2: Preparation of Single Biochar, Bamboo Fiber Biochar Carrier, and Polyurethane Biochar Carrier

Single Biochar Carrier: The bamboo biochar prepared in step (2) of Example 1 was used as the single biochar carrier.

Bamboo Fiber Biochar Carrier: 5 g of the biochar prepared in step (2) of Example 1 was added to 4 g of bamboo fibers prepared in step (1) of Example 1, and stirred at room temperature for 2 hours. Then, 240 mL of deionized water was added, and the mixture was allowed to stand and precipitate at room temperature for 2 hours. The precipitated material was then sheeted on a sheet former at 100° C. for 10 minutes to produce a paper sheet with a diameter of 20 cm. After being washed with clear water, the sheet was placed in an oven and dried at 80° C. for 12 hours. After equilibrium weighting, the bamboo fiber biochar carrier was obtained.

Polyurethane Biochar Carrier: 5 g of the biochar prepared in step (2) of Example 1 was dispersed into a polyurethane solution (consisting of 4 g of polyurethane and DMF with a DMF/PU ratio of 12.5 mL/g), and stirred at room temperature for 2 hours. Subsequently, 240 mL of deionized water was added, and the mixture was allowed to stand and precipitate at room temperature for 2 hours. The precipitated material was then sheeted on a sheet former at 100° C. for 10 minutes to produce a paper sheet with a diameter of 20 cm. After being washed with clear water, the sheet was placed in an oven and dried at 80° C. for 12 hours. After equilibrium weighting, the polyurethane biochar carrier was obtained.

The morphology results of each carrier are shown in Table 1. The single biochar carrier was in powder form, which was prone to loss during practical applications. The bamboo fiber biochar carrier was in paper form but tended to swell in fermentation media, resulting in insufficient mechanical strength. The polyurethane biochar carrier failed to form properly during preparation and could not undergo subsequent characterization. In summary, the composition of biochar, bamboo fibers, and polyurethane was necessary to maximize their respective advantages.

TABLE 1
Preparation Results of Single Biochar, Bamboo Fiber
Biochar, and Polyurethane Biochar Carriers

| Carrier | Result |
|---|---|
| Single Biochar | It was in powder form and did not form a shape. |
| Bamboo Fiber Biochar | It was shaped but had low wet strength. |
| Polyurethane Biochar | It had wet strength but did not form a shape. |

### Example 3: Testing of Tensile Strength and Burst Strength of Composite Biochar-Immobilized Microbial Fillers

### (1) Slitting

The fillers obtained from the method in Example 1 were cut into strips with 2.5 cm wide. Filler A was labeled as Sample A, Filler B as Sample B, Filler C as Sample C, and Filler D as Sample D.

### (2) Tensile Strength Testing

Each sample was placed in the testing slot of the horizontal computer-controlled tensile tester WZL-300B, with the metal clamps tightening the ends of the sample at a distance of 10 cm. The instrument was started, and the measurement was stopped after the sample broke. The tensile strength data was recorded, and the tested sample was removed. Each group of samples was tested three times, and the average value was taken. The results are shown in FIG. 2.

### (3) Burst Strength Testing

Each sample from step (1) was placed in the testing slot of the computer-controlled paper burst strength tester PN-BSM160, which had been set to a pressure of 0.3-0.5 MPa. The instrument was started, and the burst strength data of the damaged sample was recorded. Each group of materials was tested three times, and the average value was taken. The results are shown in FIG. 2.

FIG. 2 indicates that with the decrease of bamboo fibers and the increase of polyurethane, both the tensile strength and burst strength decreased to some extent, with the decrease in tensile strength being more significant. Material D had the lowest strength, Material A had the highest strength, and the strengths of Materials B and C were relatively close. During application, the tensile force experienced by the filler is relatively small, so burst strength is the main indicator to consider. Additionally, the composition of the filler also needs to comprehensively consider its adsorption, immobilization, and degradation capabilities.

### Example 4: Detection of Adsorption Capacity of Composite Biochar-Immobilized Microbial Fillers

The fillers A, B, C, and D prepared in Example 1 were each taken in an amount of 0.2 g. Using a tweezer, 0.2 g of each filler was placed into a 330 mL dry headspace vial. A 16-microliter aliquot of pure cyclohexane (12.5 mg) was injected into the vial using a syringe. After sealing, the concentration of cyclohexane in the headspace vial was measured every 5 minutes using a Fuli 9790 II gas chromatograph. Each group of samples was tested three times, and a blank control group was also set up. The fillers reached adsorption saturation within 30 minutes, and the adsorption capacities are shown in Table 2. After compositing, the fillers exhibited lower adsorption capacities but still maintained a certain level of adsorption capacity, which ensured that the concentration of the environmental substrate after immobilizing microorganisms would not be too high, thereby guaranteeing microbial activity. Among them, filler C had the highest adsorption capacity.

Gas Chromatograph Detection Conditions: The chromatographic column was KB-5 (30 m×0.32 mm×0.5 μm); the temperatures of the injector, detector (FID), and column were set to 120° C., 200° C., and 80° C., respectively; the auxiliary oven temperature was 100° C.; the split ratio was 100:1. The hydrogen flow rate was 30 mL/min, the air flow rate was 300 mL/min, the carrier gas (nitrogen) flow rate was 30 mL/min, and the gas injection volume was 1 mL.

TABLE 2
Adsorption Capacities of Composite Biochar-
Immobilized Microbial Fillers

| Substrate (62.5 mg/g) | Adsorption Capacity (mg/g) |
|---|---|
| A | 24.58 |
| B | 40.12 |
| C | 46.18 |
| D | 37.58 |

### Example 5: Detection of Immobilization Efficiency of Composite Biochar Immobilized Microbial Fillers

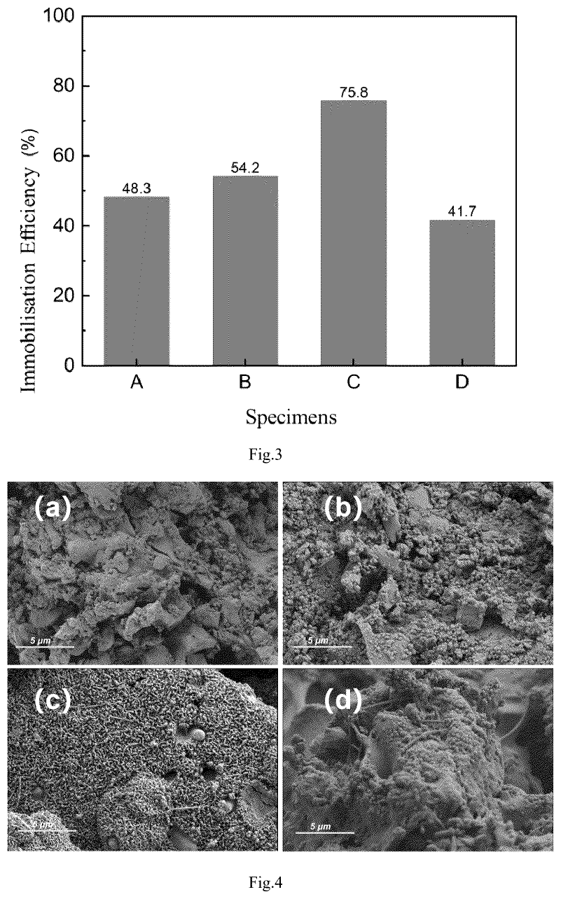

(1) Carriers A, B, C, and D, prepared according to step (4) of Example 1, were each taken in an amount of 0.2 g and sterilized in an autoclave for 20 minutes.
(2) The carriers from step (1) were placed into 50 mL of bacterial suspension with an OD600 value of 1 (denoted as OD₁) prepared according to step (3) of Example 1. They were then incubated in an incubator at 30° C. and 160 rpm for 24 hours. The culture broth was filtered, and the filter cake was the immobilized filler. The filtrate was taken to measure the bacterial suspension concentration OD₆₀₀, denoted as OD₂. The immobilization efficiency was calculated using the formula, and the results are shown in FIG. 3. With the increase of polyurethane and the decrease of bamboo fiber, the immobilization efficiency first increased and then decreased, with filler C achieving the maximum of 75.8%. This indicates that the addition of polyurethane has a certain impact on the comprehensive performance of the filler. When the amount of polyurethane added is low, microorganisms are prone to elution, resulting in poor immobilization. When too much polyurethane is added, it may inhibit microbial activity, leading to the worst immobilization effect.

$$Immobilization Efficiency = ( {OD}_{1} - {OD}_{2} ) / {OD}_{1} \times 100 \% .$$

(3) The immobilized fillers from step (2) were air-dried at room temperature for morphological observation. Fillers A and B exhibited fiber swelling due to the excessive addition of bamboo fiber, and some biochar loss led to an overall slight whitening. In contrast, fillers C and D maintained their original morphology after immobilization.
(4) The immobilized fillers from step (2) were added to 1 mL of PBS buffer (0.05 M, pH 7) containing 2.5 g/L glutaraldehyde and fixed at 4° C. for 12 hours. Residual glutaraldehyde was washed away with PBS twice. The samples were then dehydrated with ethanol-water solutions of different volume concentrations (30%, 50%, 70%, 90%, 100%), with each step lasting for 10 to 15 minutes. The dehydrated samples were vacuum freeze-dried at 60° C. After sputtering with gold, their distribution was observed under a scanning electron microscope. The scanning electron microscope images are shown in FIG. 4. The results indicate that microorganisms were well attached to the surface and interior of the fillers. The level of microbial growth was consistent with the above immobilization efficiency results. Among them, groups B (mass ratio of BF to PU was 3:2) and C (mass ratio of BF to PU was 2:3) exhibited good microbial growth effects.

In summary, the preferred mass ratio of BF to PU is 3:2.

### Example 6: Degradation of VOCs by Composite Biochar Immobilized Microbial Fillers

0.2 g of filler A, filler B, filler C, and filler D, each prepared by the method of Example 1, were taken and placed into 330 mL headspace vials containing 50 mL of inorganic salt medium using a tweezer. 12 microliters of pure cyclohexane (with a concentration of approximately 189.84 mg/L in the liquid phase) was injected into each vial using a syringe, and the vials were then sealed. The concentration of cyclohexane in the headspace vials was measured every 5 minutes using the method described in Example 3.

Under the same conditions, a blank control was set up without the addition of any filler, and a free bacteria control was established by adding 1 mL of a bacterial suspension with an OD600 value of 0.5, prepared by the method of Example 1.

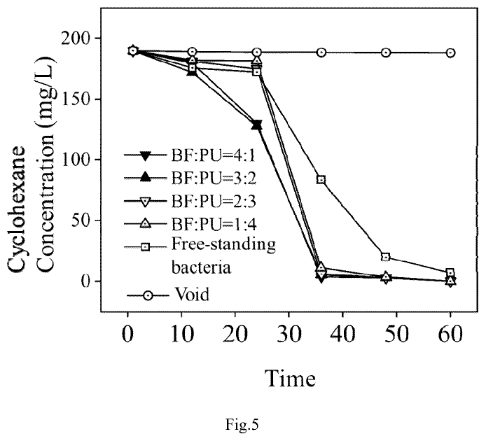

The results, as shown in FIG. 5, indicated that there was little difference in degradation efficiency between the immobilized fillers and the free bacteria within 60 hours, but the immobilized fillers degraded faster, with almost complete degradation achieved within 36 hours. This may be attributed to the good protective effect of the immobilized fillers on the microorganisms and their better mass transfer effect for the substrate. The immobilized fillers with a bamboo fiber to polyurethane mass ratio of 4:1 and 3:2 had shorter degradation initiation periods than the other two fillers, which may be due to the excessive addition of polyurethane in the other two fillers, having an adverse effect on the microorganisms.

1. A composite biochar immobilized microbial filler, wherein it is made with bamboo fiber, thermoplastic polyurethane, and bamboo biochar as the carrier, and an efficient degrading bacterial suspension for volatile organic pollutants as the active microorganism; wherein the mass ratio of bamboo biochar to bamboo fiber and thermoplastic polyurethane is 5:1-4:1-4; the OD₆₀₀ of the efficient degrading bacterial suspension is 0.1-1.0, and the volume usage is 100-250 mL/g based on mass of the carrier.

2. The composite biochar immobilized microbial filler as claimed in claim 1, wherein the mass ratio of bamboo biochar to bamboo fiber and thermoplastic polyurethane is 5:2:3, the OD₆₀₀ of the efficient degrading bacterial suspension is 0.5, and the volume usage is 125 mL/g based on the mass of the carrier.

3. The composite biochar immobilized microbial filler as claimed in claim 1, wherein the efficient degrading bacterium is Acinetobacter venetianus with the deposit number CCTCC NO: M 2022719, which degrades cyclohexane.

4. The composite biochar immobilized microbial filler as claimed in claim 1, wherein the efficient degrading bacterial suspension is obtained as follows: inoculating the efficient degrading bacteria into LB solid medium, culturing them at 30° C. for 1 day, then inoculating them into a fermentation medium, incubating them at 30° C. and 160 rpm for 48 hours, after centrifugation, collecting wet bacterial cells, washing them with a buffer solution, and then resuspending them with sterile water to obtain the bacterial suspension; wherein the fermentation medium is composed of: 5 g/L yeast extract, 10 g/L NaCl, 10 g/L peptone, pH natural, and deionized water as the solvent.

5. The composite biochar immobilized microbial filler as claimed in claim 1, wherein the bamboo fiber is prepared using the following method: soaking the bamboo fiber feedstock in distilled water at room temperature for 6-12 hours, washing it with distilled water, then adding distilled water to the soaked material and pouring the mixture into a beater for beating, taking the slurry with a beating degree of 30-70° SR and drying it to obtain bamboo fiber.

6. The composite biochar immobilized microbial filler as claimed in claim 1, wherein the bamboo biochar is prepared using the following steps: placing the absolutely dry bamboo material in a porcelain boat in a tube furnace, under a nitrogen atmosphere, increasing the temperature to 500-700° C. at a rate of 2-6° C./min and maintaining it for 1-3 hours, stopping nitrogen supply, then using water vapor to increase the temperature to 600-1000° C. at a rate of 1-4° C./min and maintaining it for 1-3 hours, cooling the material to room temperature, and sieving it through an 80-100 mesh screen to obtain bamboo biochar.

7. A method for preparing the composite biochar immobilized microbial filler as claimed in claim 1, comprising the following steps:
(1) dissolving the thermoplastic polyurethane in N,N-dimethylformamide with stirring at room temperature to yield a polyurethane solution; dispersing bamboo biochar and bamboo fiber into the polyurethane solution, stirring at room temperature for 1-3 hours, then adding deionized water, allowing the mixture to stand for precipitation at room temperature, then taking the precipitate, pressing it into shape, and drying it to obtain a carrier;
(2) immersing the carrier from step (1) in the efficient degrading bacterial suspension, and subjecting it to immobilization on a shaker at 25-35° C. and 100-200 r/min for 20-30 hours; after removal, drying it at room temperature to obtain the composite biochar immobilized microorganism.

8. The method as claimed in claim 7, wherein in step (1), the volume usage of N,N-dimethylformamide is 10-15 mL/g, based on the mass of polyurethane; the volume usage of deionized water is 50-275 mL/g, based on the mass of polyurethane; pressing into shape and drying are carried out as follows: the precipitate is manufactured on the sheet former at 100° C. for 10 minutes, and then placed in an oven and dried at 80° C. for 12 hours to obtain a paper sheet.

9. An application of the composite biochar immobilized microbial filler as claimed in claim 1 in the degradation of one or more volatile organic pollutants.

10. The application as claimed in claim 9, wherein the application method is as follows: using the composite biochar immobilized microbial filler as the catalyst, one or more volatile organic pollutants as the substrate, and an inorganic salt culture medium as the reaction medium, and carry out the reaction under conditions of a pH range of 5-8 and a temperature range of 25-35° C., to achieve the degradation of volatile organic pollutants; wherein the amount of the catalyst used is 2-6 g/L, based on the volume of the inorganic salt culture medium; the concentration of the volatile organic pollutant is 150-300 mg/L, based on the volume of the inorganic salt culture medium; and the volatile organic pollutant is cyclohexane.
